(19) United States
(12) Patent Application Publication
(10) Pub. No.: US 2026/0337848 A1
(43) Pub. Date: Oct. 29, 2026

(54) TEXTILE WASHING METHOD

(71) Applicant: HENKEL AG & CO. KGAA, Düsseldorf (DE)

(72) Inventors: Arndt KESSLER, Monheim (DE); Ingo HARDACKER, Hamminkeln (DE); Christian DEGERING, Erkrath (DE); Susanne WIELAND, Dormagen (DE); Robert RUIZ HERNANDEZ, Düsseldorf (DE); Sergio LINZ, Köln (DE)

(21) Appl. No.: 19/109,813

(22) PCT Filed: Jul. 19, 2023
(86) PCT No.: PCT/DE2023/100533

§ 371 Date: Mar. 7, 2025

(30) Foreign Application Priority Data
Sep. 7, 2022 (EP) 22194253.5

(51) Int. Cl. D06F 33/57; C11D 1/04; C11D 1/14; C11D 1/22; C11D 1/72; C11D 1/83; C11D 3/386; C11D 3/43; D06F 103/20; D06F 105/42; D06L 1/12

(52) U.S. Cl. CPC D06F 33/57; C11D 1/83; C11D 3/38618; C11D 3/43; C12Y 304/21062; D06L 1/12; C11D 1/04; C11D 1/143; C11D 1/22; C11D 1/72; D06F 2103/20; D06F 2105/42

(57) ABSTRACT

A method for washing textiles in a washing machine is described. A washing machine is provided with a wash program, including a main wash cycle with the duration t_{w}. Textiles are introduced into the laundry treatment chamber of the washing machine. An aqueous liquor is introduced into the laundry treatment chamber of the washing machine. A first detergent composition is introduced into the laundry treatment chamber of the washing machine during the main wash cycle at a point in time of from 0 to 10% of the duration t_{w}. The first detergent composition includes at least one surfactant. A second detergent composition is introduced into the into the laundry treatment chamber of the washing machine during the main wash cycle at a point in time of from 11 to 99% of the duration t_{w}. The second detergent composition includes i) at least 4 wt. % of an active protease protein and ii) at least 80 wt. % of a liquid carrier.

The present invention relates to a textile washing method. In particular, the application relates to a multi-stage machine textile washing method in the course of which washing and cleaning-active substances are metered in a staggered manner.

While less than one third of people in total currently have access to a textile washing machine, in some regions of the world machine textile cleaning has been the standard method for removing dirt and for refreshing laundry since the 1970s.

Over recent decades, both machine technology and the detergents used in machine-based textile cleaning methods have been continuously developed and improved in terms of their performance and their ecological footprint. While development efforts were initially focused on improving the individual components of the washing process, for example the textile washing machine and its mechanical aspects and programs or the textile detergent, for some time now the focus of interest has also been on improving the interaction between these components.

The international application WO 2012/048911 A1 describes a washing method using a textile washing machine, in the course of which the textiles are sprayed with a rinsing solution in a rinse cycle.

In European Patent EP 3 428 336 B1, washing methods with a minimum duration of 110 minutes are disclosed, in the course of which detergents containing rejuvenating agents are added to the washing liquid.

European Patent EP 2 711 413 B1 relates to washing methods that are characterized by a staggered metering of different washing-active substances.

European Patent EP 2 566 943 B1 discloses the sensor-controlled, staggered metering of washing-active substances, for example peroxycarboxylic acids, into the interior of a textile washing machine.

Against the background of the previous developments, there is also the technical object of improving the washing performance of textile detergents in textile washing machines.

In order to achieve this object, the invention proposes a method for washing textiles in a drum washing machine, having the steps of
a) providing a washing machine with a wash program, including a main wash cycle with the duration t_{w};
b) introducing textiles into the laundry treatment chamber of the washing machine;
c) introducing an aqueous liquor into the laundry treatment chamber of the washing machine;
d) introducing a first detergent composition, comprising at least one surfactant, into the laundry treatment chamber of the washing machine during the main wash cycle at a point in time of 0 to 10% t_{w};
e) introducing a second detergent composition comprising, based on the total weight thereof,
   i) at least 4 wt. % of active protease protein
   ii) at least 80 wt. % of a liquid carrier
into the aqueous liquor of the main wash cycle at a point in time of 11 to 99% t_{w}.

A motor-driven device for cleaning textiles is referred to as a washing machine. Particularly preferred are rotary washing machines with an inner tub rotatable about a horizontal axis. The method according to the invention is suitable in particular for being carried out in a drum washing machine with an outer tub, an inner tub attached within the outer tub, as a laundry treatment chamber, and a pumping device that is configured to pump aqueous liquor out of the outer tub.

In step a) of the method, a washing machine with a wash program, including a main wash cycle with the duration t_{w}, is provided. Conventional washing machines usually have a plurality of wash programs provided for cleaning different textiles, which wash programs can have, in addition to a main wash cycle, pre-rinse, rinse and/or spin cycles. Preferred wash programs comprise a main wash cycle, at least one rinse cycle and at least one spin cycle. Alternative wash programs have at least one pre-wash cycle, a main wash cycle, at least one rinse cycle, and at least one spin cycle.

In addition to the mechanical forces acting on the laundry, the detergent used and the liquor temperature reached in the washing liquor, the duration of the wash cycle, in particular of the main wash cycle, has an influence on the cleaning performance achieved. The duration t_{w} of the main wash cycle used in the washing method is preferably 15 to 400 minutes, preferably 30 to 240 minutes, and in particular 60 to 180 minutes.

The textiles introduced into the laundry treatment chamber in step b) can be, for example, cotton or synthetic textiles, but also mixed fabrics.

In a preferred embodiment of the washing method, the drum washing machine has a device for detecting the weight of the textiles introduced in step b).

In one exemplary embodiment, the drum washing machine has a load sensor as a device for detecting the textile weight. The use of such a sensor makes it possible to directly ascertain in step b) the weight of the textiles introduced into the laundry treatment chamber of the washing machine.

In a preferred embodiment of the method, the weight of the textiles introduced into the laundry treatment chamber of the washing machine in step b) is ascertained by means of a load sensor and a treatment parameter is determined on the basis of this weight information and, if applicable, further information, preferably information detected by sensors. The treatment parameter can, for example, be the duration of the washing program or its program steps such as the pre-wash cycle, main wash cycle or rinse cycle. Alternatively, the treatment parameter relates to the temperature of the aqueous liquor, preferably the temperature of the aqueous liquor in the main wash cycle. It is particularly preferred if the treatment parameter relates to the amount of the first or second detergent composition introduced in step d) and/or e).

Preferably, the washing program is configured such that the textile weight detected by means of the device influences the amount of the second detergent composition introduced in step e).

The treatment parameter can be determined by comparing the determined textile weight with reference values and the treatment parameters assigned to these reference values. Corresponding reference values can be stored in a database.

The determined treatment parameter can be made available to the user. The treatment parameter can be output to the user on a display, or a corresponding output can be triggered, for example an output on a screen, particularly in text form, and/or an acoustic output such as a verbal notification. The user can then carry out the treatment. Alternatively or additionally, in one embodiment of the method, the treatment parameter can be output to the washing machine. For example, the treatment parameter may represent at least one parameter of the washing program that is output to the washing machine, such that the washing machine for example adopts the corresponding washing program as a preset and the user merely has to start the cleaning device. It is equally conceivable for the washing machine to automatically carry out the washing program with the output. The washing machine may for example have a metering device for detergent compositions, in order to automatically provide the amount of the first and/or second detergent composition. This makes the method more user-friendly as a result.

The aqueous liquor introduced into the laundry treatment chamber in step c) preferably has a volume of 3 to 40 l, preferably 6 to 30 l, and in particular 8 to 20 l.

Since the washing method according to the invention achieves above-average cleaning results, particularly in the case of high water hardness, the water hardness of the aqueous liquor introduced in step c) is preferably more than 14° dH, preferably more than 17°dH and in particular more than 21° dH.

In a preferred embodiment of the washing method, the drum washing machine has a device for detecting the water hardness of the aqueous liquor used in step c). In one exemplary embodiment, the drum washing machine has a sensor for determining the water hardness as a device for determining the water hardness. The use of such a sensor makes it possible to directly determine the hardness of the aqueous liquor introduced into the laundry treatment chamber of the washing machine in step c).

In an alternative embodiment, the hardness of the aqueous liquor introduced into the laundry treatment chamber of the washing machine in step c) is determined indirectly. An indirect route to determining water hardness is, for example, determining the geographical location of the drum washing machine and comparing this location data with water hardnesses stored in a database for different locations. The location data can be entered by the user, for example in the form of coordinates or information equivalent to coordinates, such as zip codes, or can be determined automatically by the washing machine itself, for example through a GPS positioning system.

In a preferred embodiment of the method, the water hardness of the aqueous liquor introduced into the laundry treatment chamber of the washing machine in step c) is determined by means of a device for determining water hardness, and a treatment parameter is determined on the basis of this water hardness information and, if applicable, further information, preferably information acquired by sensors. The treatment parameter can, for example, be the duration of the washing program or its program steps such as the pre-wash cycle, main wash cycle or rinse cycle. Alternatively, the treatment parameter relates to the temperature of the aqueous liquor, preferably the temperature of the aqueous liquor in the main wash cycle. It is particularly preferred if the treatment parameter relates to the amount of the first or second detergent composition introduced in step d) and/or e).

Preferably, the washing program is configured such that the water hardness detected by means of the device influences the amount of the second detergent composition introduced in step e).

The treatment parameter can be determined by comparing the determined water hardness with reference values and the treatment parameters assigned to these reference values. Corresponding comparative values can be stored in a database.

The determined treatment parameter can be made available to the user. The treatment parameter can be output to the user on a display, or a corresponding output can be triggered, for example an output on a screen, particularly in text form, and/or an acoustic output such as a verbal notification. The user can then carry out the treatment. Alternatively or additionally, in one embodiment of the method, the treatment parameter can be output to the washing machine. For example, the treatment parameter may represent at least one parameter of the washing program that is output to the washing machine, such that the washing machine for example adopts the corresponding washing program as a preset and the user merely has to start the cleaning device. It is equally conceivable for the washing machine to automatically carry out the washing program with the output. The washing machine may for example have a metering device for detergent compositions, in order to automatically provide the amount of the first and/or second detergent composition. This makes the method more user-friendly as a result.

A preferred subject-matter of the application is a method for washing textiles in a drum washing machine, having the steps of
a) providing a washing machine with a wash program, including a main wash cycle with the duration t_{w};
b) introducing textiles into the laundry treatment chamber of the washing machine;
c) introducing an aqueous liquor into the laundry treatment chamber of the washing machine;
d) introducing a first detergent composition, comprising at least one surfactant, into the laundry treatment chamber of the washing machine during the main wash cycle at a point in time of 0 to 10% t_{w};
e) introducing a second detergent composition comprising, based on the total weight thereof,
   i) at least 4 wt. % of active protease protein
   ii) at least 80 wt. % of a liquid carrier
into the aqueous liquor of the main wash cycle at a point in time of 11 to 99% t_{w},

wherein the water hardness of the aqueous liquor introduced into the laundry treatment chamber of the washing machine in step c) is determined by means of a device for determining the water hardness, and a treatment parameter is determined by comparing the determined water hardness with reference values and treatment parameters assigned to these reference values, which treatment parameter relates to the amount of the first or second detergent composition introduced in step d) and/or step e), and the first detergent composition is introduced in step d), and/or the second detergent composition is introduced in step e), in an amount corresponding to the determined treatment parameter.

A particularly preferred subject matter of the application is a method for washing textiles in a drum washing machine, having the steps of
a) providing a washing machine with a wash program, including a main wash cycle with the duration t_{w};
b) introducing textiles into the laundry treatment chamber of the washing machine;
c) introducing an aqueous liquor into the laundry treatment chamber of the washing machine;
d) introducing a first detergent composition, comprising at least one surfactant, into the laundry treatment chamber of the washing machine during the main wash cycle at a point in time of 0 to 10% t_{w};
e) introducing a second detergent composition comprising, based on the total weight thereof,
   i) at least 4 wt. % of active protease protein
   ii) at least 80 wt. % of a liquid carrier
into the aqueous liquor of the main wash cycle at a point in time of 11 to 99% t_{w},

wherein the water hardness of the aqueous liquor introduced into the laundry treatment chamber of the washing machine in step c) is determined by means of a device for determining the water hardness, and a treatment parameter is determined by comparing the determined water hardness with reference values and treatment parameters assigned to these reference values, which treatment parameter relates to the amount of the second detergent composition introduced in step e), and the second detergent composition is introduced in step e) in an amount corresponding to the determined treatment parameter.

The aqueous liquor preferably has a temperature T₁ of 18 to 25 °C in step c).

The loading of the washing machine with textiles in step b) and the volume of the aqueous liquor introduced in step c) are preferably coordinated with one another in such a way that the weight ratio of aqueous liquor to textiles in step c) is above 1:1, preferably above 2:1, and in particular above 5:2.

The first detergent composition is introduced into the laundry treatment chamber at a point in time of 0 to 10% t_{w} in step d). In other words, the first detergent composition is already located in the laundry treatment chamber at the start of the main wash cycle (point in time of 0 t_{w}) or is introduced into the laundry treatment chamber within a period of 10% of the duration t_{w} of the main wash cycle.

In a first method variant, the aqueous liquor is introduced into the laundry treatment chamber of the washing machine before the first detergent composition. Such a method variant can be realized, for example, by means of a pre-rinse cycle, in the course of which the textiles are pre-rinsed and/or soaked with water but not yet cleaned by means of the actual detergent composition.

A second method variant provides the simultaneous introduction of the aqueous liquor and the first detergent composition into the laundry treatment chamber of the washing machine. If the aqueous liquor is conducted into the laundry treatment chamber without a pre-rinse cycle, for example through the washing machine dispensing drawer filled with the first detergent composition, the aqueous liquor and the first detergent composition are introduced into the laundry treatment chamber simultaneously.

Finally, it is also possible for the aqueous liquor to be introduced into the laundry treatment chamber of the washing machine after the first detergent composition, for example by applying the first detergent composition directly to the textiles in pre-portioned form or by means of a metering aid before the start of the wash program.

The surfactant-containing first detergent composition used in step d) is preferably highly concentrated and, based on the total weight thereof, comprises
i) 50 to 95 wt. % surfactant
ii) less than 10 wt. % water.

In particularly preferred embodiments, the surfactant-containing first detergent composition contains 55 to 92 wt. %, in particular 60 to 88 wt. % surfactant.

The surfactant-containing first detergent composition used in step d) is preferably metered from a container integrated into the washing machine, which container is filled with several times the amount of first detergent composition required to run a washing program.

The group of surfactants includes the non-ionic, anionic, cationic and amphoteric surfactants. The compositions according to the invention can comprise one or more of the surfactants mentioned. Particularly preferred compositions contain surfactant from the group of anionic and non-ionic surfactants.

The anionic surfactant is preferably selected from the group comprising C₉₋₁₃ alkylbenzenesulfonic acids, olefin sulfonic acids, C₁₂₋₁₈ alkanesulfonic acids, ester sulfonic acids, alk(en)yl sulfonic acids, fatty alcohol ether sulfonic acids and mixtures thereof. Compositions which comprise C₉₋₁₃ alkylbenzenesulfonic acids and fatty alcohol ether sulfonic acids as the anionic surfactant have particularly good dispersing properties. Surfactants of the sulfonic acid type that can be used are preferably C₉₋₁₃ alkylbenzenesulfonic acids, olefin sulfonic acids, i.e. mixtures of alkene and hydroxyalkane sulfonic acids, and disulfonic acids, as are obtained, for example, from C₁₂₋₁₈ monoolefins having a terminal or internal double bond by way of sulfonation with gaseous sulfur trioxide and subsequent alkaline or acid hydrolysis of the sulfonation products. C₁₂₋₁₈ alkanesulfonic acids and the esters of α-sulfofatty acids (ester sulfonic acids) are also suitable, for example the α-sulfonated methyl esters of hydrogenated coconut, palm kernel or tallow fatty acids.

In particularly preferred washing methods, the first detergent composition contains anionic surfactant from the group of C₈₋₁₈ alkylbenzenesulfonic acids. The use of alkylbenzenesulfonic acids from the group of C₉₋₁₅ alkylbenzenesulfonic acids, in particular C₉₋₁₃ alkylbenzenesulfonic acids, is particularly preferred.

Alternatively, but preferably in combination with the alkylbenzenesulfonic acid, the first detergent composition comprises anionic surfactant from the group of alkyl ether sulfonic acids.

Preference is given to alkyl ether sulfonic acids having the formula R₁—O-(AO)ₙ—SO₃H, wherein
R₁ represents a linear or branched, substituted or unsubstituted alkyl radical, preferably a linear, unsubstituted alkyl radical, particularly preferably a fatty alcohol radical. Preferred radicals R₁ are selected from decyl, undecyl, dodecyl, tridecyl, tetradecyl, pentadecyl, hexadecyl, heptadecyl, octadecyl, nonadecyl and eicosyl radicals and mixtures thereof, the representatives with an even number of C atoms being preferred. Particularly preferred radicals R₁ are derived from fatty alcohols having 12 to 18 C atoms, for example from coconut fatty alcohol, tallow fatty alcohol, lauryl alcohol, myristyl alcohol, cetyl alcohol or stearyl alcohol, or from oxo alcohols having 10 to 20 C atoms. AO represents an ethylene oxide (EO) or propylene oxide (PO) group, preferably an ethylene oxide group. The index n in the formula is an integer from 1 to 50, preferably from 1 to 20, and in particular from 2 to 10. Very particularly preferably, n is 2, 3, 4, 5, 6, 7 or 8.

As a further optional component which is preferably present in the first detergent composition in combination with the aforementioned alkylbenzenesulfonic acids and alkyl ether sulfonic acids, the first detergent composition contains anionic surfactant from the group of fatty acids. Particularly preferred fatty acids are selected from the group of caprylic acid, capric acid, lauric acid, myristic acid, palmitic acid, stearic acid, oleic acid, linoleic acid and mixtures thereof.

Preferred first detergent compositions used in step d) of the method contain, based on the total weight thereof, 30 to 90 wt. %, preferably 40 to 80 wt. %, and in particular 45 to 70 wt. % anionic surfactant. The proportion by weight of the anionic surfactant is determined as the proportion by weight of the anionic surfactant acid, regardless of the degree of neutralization of the anionic surfactant in the detergent composition.

As a further optional component, the first detergent composition contains nonionic surfactant, preferably nonionic surfactant from the group of the ethoxylated primary C6-18 alcohols, preferably the ethoxylated primary C6-18 alcohols having a degree of alkoxylation ≥2, particularly preferably the C12-14 alcohols having 4 EO or 7 EO, the C9-11 alcohols having 7 EO, the C13-15 alcohols having 5 EO, 7 EO or 8 EO, the C13-15 oxo alcohols having 7 EO, the C12-18 alcohols having 5 EO or 7 EO, in particular the C12-18 fatty alcohols having 7 EO or the C13-15 oxo alcohols having 7 EO.

Preferred first detergent compositions used in step d) of the method contain, based on the total weight thereof, 7 to 50 wt. %, preferably 12 to 40 wt. %, and in particular 15 to 30 wt. % nonionic surfactant.

A group of optional constituents of the first detergent composition is formed by the polyalkoxylated amines having a weight-average molecular weight Mw in the range from 600 g/mol to 10 000 g/mol, which are obtained by reacting ammonia or primary alkylamines or hydroxyalkylamines having a molecular weight of less than 200 g/mol with alkylene oxides. The proportion by weight of these polyalkoxylated amines with respect to the total weight of the first detergent composition is preferably 0.5 to 10 wt. %, particularly preferably 1 to 8 wt. % and in particular 2 to 6 wt. %.

Preferred polyalkoxylated amines have a weight-average molecular weight MW in the range from 1300 g/mol to 6000 g/mol, in particular from 1400 g/mol to 4500 g/mol. (The average molecular weights indicated here, and later possibly for other polymers, are weight-average molecular weights MW which can in principle be determined by means of gel permeation chromatography with the aid of an RI detector, the measurement expediently being carried out against an external standard.) For their preparation, it is possible to start, in a known manner, from ammonia, a monoalkylamine, a monoalkyl-monoalkanolamine or a monoalkyl-dialkanolamine or a monoalkanolamine, dialkanolamine or trialkanolamine, for example triethanolamine, methyl-, ethyl-, propyl- and isopropyl-diethanolamine, methyl-, ethyl-, propyl- and isopropyl-diisopropanolamine, tripropanolamine, triisopropanolamine, N,N-Di-(2-hydroxyethyl)cyclohexylamine, N,N-Di-(2-hydroxypropyl)cyclohexylamine, n-butylamine, n-hexylamine, n-octylamine, isopropylamine, sec-butylamine, tert-butylamine, cyclohexylamine, 2-ethylhexylamine, 2-phenylethylamine and mixtures thereof, which is reacted with an alkylene oxide, in particular selected from the group consisting of ethylene oxide, propylene oxide, butylene oxide and mixtures thereof, in particular with a mixture containing propylene oxide and preferably ethylene oxide, particularly preferably with propylene oxide. The polyalkoxylated amines thus obtained may be block or random structures. Particular preference is given, inter alia, to a polyalkoxylated amine obtainable by propoxylation of triethanolamine, preferably having a length of the three side arms of 15 propylene oxide units in each case. Also preferred is a polyalkoxylated amine obtainable by propoxylation of triisopropanolamine, preferably having a length of the three side arms of 15 propylene oxide units in each case. Polyalkoxylated monoalkylamines having a linear, branched or cyclic alkyl group are likewise suitable, an alkylene oxide selected from the group consisting of ethylene oxide, propylene oxide, butylene oxide and mixtures thereof being alkoxylated, preferably with a mixture comprising propylene oxide, more preferably with propylene oxide. Preference is also given to a polyalkoxylated amine obtainable by propoxylation of tert-butylamine, preferably having a length of the two side arms of 12 propylene oxide units in each case.

Preferred polyalkoxylated amines satisfy the general formula

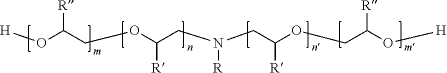

in which R represents a linear, optionally branched or optionally cyclic alkyl group having 1 to 12 C atoms or a group —(CH₂CHR′O)_{n″}—(CH₂CHR″O)_{m″}—H group,
R′ and R″, independently of one another, represent H, CH₃ or CH₂CH₃,
n, n′ and n″ represent, independently of one another, numbers from 0 to 30, preferably from 0 to 10 and in particular 0 to 5, and
m, m′ and m″ represent, independently of one another, numbers from 0 to 30, preferably from 5 to 20 and in particular from 12 to 16,
with the proviso that the sum n+n′+n″+m+m′+m″ is at least 14, preferably in the range of from 18 to 100 and in particular in the range of from 20 to 70. Preferably, in the compounds of the formula I, at least one of the functional groups R′ and R″ is a CH₃ group.

As a further optional component, the first detergent composition contains a polyalkoxylated polyalkylene imine, which can be obtained by reacting polyalkylene imines with alkylene oxides, with proportions by weight based on the total weight of the first detergent composition of 0.5 to 10 wt. %, preferably 1 to 8 wt. % and in particular 2 to 6 wt. % being preferred.

The polyalkoxylated polyalkylene imine is a polymer having a polyalkylene imine backbone which carries polyalkoxy groups on the N atoms. It preferably has a weight-average molecular weight Mw in the range from 5000 g/mol to 60000 g/mol, in particular from 10000 g/mol to 22500 g/mol. The polyalkylene imine has primary amino functions at the ends and preferably both secondary and tertiary amino functions in the interior, and optionally it can also have only secondary amino functions in the interior such that the result is not a branched-chain but a linear polyalkylene imine. The ratio of primary to secondary amino groups in the polyalkylene imine is preferably in the range from 1:0.5 to 1:1.5, in particular in the range from 1:0.7 to 1:1. The ratio of primary to tertiary amino groups in the polyalkylene imine is preferably in the range from 1:0.2 to 1:1, in particular in the range from 1:0.5 to 1:0.8. The polyalkylene imine preferably has a weight-average molecular weight in the range from 500 g/mol to 50000 g/mol, in particular from 550 g/mol to 2000 g/mol. The N atoms in the polyalkylene imine are preferably separated from one another by alkylene groups having 2 to 12 C atoms, in particular 2 to 6 C atoms, although it is not necessary for all the alkylene groups to have the same number of C atoms. Ethylene groups, 1,2-propylene groups, 1,3-propylene groups and mixtures thereof are particularly preferred. The primary amino functions in the polyalkylene imine can carry 1 or 2 polyalkoxy groups and the secondary amino functions can carry 1 polyalkoxy group, not every amino function having to be alkoxy group-substituted. The average number of alkoxy groups per primary and secondary amino function in the polyalkoxylated polyalkylenimine is preferably 5 to 100, in particular 10 to 50. The alkoxy groups in the polyalkoxylated polyalkylene imine are preferably ethoxy, propoxy or butoxy groups or mixtures thereof. Polyethoxylated polyethyleneimines are particularly preferred. The polyalkoxylated polyalkylene imines are obtainable by reacting the polyalkylene imines with epoxides corresponding to the alkoxy groups. If desired, the terminal OH function of at least some of the polyalkoxy substituents can be replaced by an alkyl ether function having 1 to 10, in particular 1 to 3, C atoms.

Detergent compositions with the lowest possible proportion of inactive ingredients are preferably provided for use in the washing method according to the invention. Consequently, it is preferred if the first detergent composition contains less than 10 wt. %, preferably less than 6 wt. %, and in particular less than 2 wt. %, water. As the proportion of inactive ingredients decreases, not only does the amount of resources required for packaging, storage and transportation of the first detergent composition decrease, but at the same time the volume of first detergent composition used per washing method also decreases and thus the scope of a storage container, for example a storage container integrated into the washing machine, increases.

For the handleability of the first detergent composition, in particular the dosing thereof, a proportion by weight of 0.5 to 29 wt. %, preferably 4.5 to 27 wt. % and in particular 10 to 24 wt. % of organic solvent, based on the total weight of the first detergent composition, has proven to be advantageous. Cleaning-enhancing effects in the washing method can be attributed to organic solvents. This applies in particular when using organic solvents from the group of organic amines, preferably monoethanol amine. The use thereof not only has a cleaning-enhancing effect, but also enables the partial or complete neutralization of any anionic surfactant acids present in the first detergent composition.

In a preferred embodiment of the washing method, the first detergent composition comprises anionic surfactant from the group of C8-18 alkylbenzenesulfonic acids, alkyl ether sulfonic acids and fatty acids, and also a superstoichiometric amount, relative to the anionic surfactant, of organic amine, preferably monoethanol amine.

In summary, washing methods are preferred in which the first detergent composition in step d) contains, based on the total weight thereof
i) 30 to 90 wt. %, preferably 40 to 80 wt. %, and in particular 45 to 70 wt. % anionic surfactant from the group of C₈₋₁₈ alkylbenzenesulfonic acids, alkyl ether sulfonic acids and fatty acids,
ii) 7 to 50 wt. %, preferably 12 to 40 wt. %, and in particular 15 to 30 wt. % nonionic surfactant from the group of ethoxylated primary C6-18 alcohols,
iii) 0.5 to 29 wt. %, preferably 4.5 to 27 wt. %, and in particular 10 to 24 wt. % organic solvent from the group of organic amines,
iv) less than 10 wt. %, preferably less than 6 wt. % and in particular less than 2 wt. % water.

In a method variant, the first detergent composition is preferably introduced into the laundry treatment chamber of the washing machine at a point in time of 0 t_{w} in step d).

More preferably, the first detergent composition is introduced into the laundry treatment chamber of the washing machine at a point in time of >0 to 10% t_{w}, preferably >0 to 5% t_{w}, in step d).

In contrast, in step e), the second detergent composition is introduced into the aqueous liquor at a point in time of 20 to 70% t_{w}, preferably at a point in time of 25 to 65% t_{w}, particularly preferably at a point in time of 30 to 60% t_{w}.

The second detergent composition is highly concentrated in terms of its content of active protease enzyme and comprises, based on the total weight thereof, at least 4 wt. % active protease protein and at least 80 wt. % of a liquid carrier.

Proteases are among the technically most important enzymes. For washing and cleaning agents, they are the longest established enzymes and are contained in virtually all modern, high-performance washing and cleaning agents. They cause the degradation of protein-containing stains on the articles to be cleaned. In turn, proteases of the subtilisin type (subtilases, subtilopeptidases, EC 3.4.21.62) are particularly important, which proteases are serine proteases due to the catalytically active amino acids. They act as unspecific endopeptidases and hydrolyze any acid amide bonds within peptides or proteins. Their optimum pH is usually in the distinctly alkaline range. Subtilases are naturally formed by microorganisms. In particular, the subtilisins formed and secreted by the Bacillus species are the most significant group of subtilases.

Examples of subtilisin-type proteases that are preferably used in washing and cleaning agents are the subtilisins BPN′ and Carlsberg, protease PB92, subtilisins 147 and 309, the alkaline protease from Bacillus lentus, in particular Bacillus lentus DSM 5483, subtilisin DY and the enzymes thermitase, proteinase K and proteases TW3 and TW7, which are to be classified as subtilases, but no longer as subtilisins in the narrower sense, as well as variants of said proteases that have an amino acid sequence which is altered compared to the starting protease. Proteases are altered in a targeted or random manner by methods known from the prior art and are thus optimized for use in washing and cleaning agents, for example. This includes point, deletion or insertion mutagenesis, or fusion with other proteins or protein parts. Thus, appropriately optimized variants are known for most proteases known from the prior art.

In preferred embodiments of the method, the second detergent composition comprises, based on the total weight thereof, 4 to 18 wt. %, preferably 5 to 15 wt. % and in particular 7 to 12 wt. % active protease protein. The concentration of active protease protein in the second detergent composition is thus 10 to 40 times higher than the concentration of active protease protein in usual commercially available detergent compositions.

Preferred proteases comprise
a) a protease that has proteolytic activity and comprises an amino acid sequence which is at least 70% identical, and with increasing preference at least 71%, 72%, 73%, 74%, 75%, 76%, 77%, 78%, 79%, 80%, 81%, 82%, 83%, 84%, 85%, 86%, 87%, 88%, 89%, 90.5%, 91%, 91.5%, 92%, 92.5%, 93%, 93.5%, 94%, 94.5%, 95.5%, 96%, 96.5%, 97%, 97.5%, 98%, 98.5% or 98.8% identical to the amino acid sequence given in SEQ ID NO:1 over its entire length and, in each case based on the numbering according to SEQ ID NO:3 has, (i) at the position corresponding to position 101, the amino acid substitution R101E, and (ii) at at least one of the positions corresponding to positions 3, 4, 45, 55, 58, 59, 61, 87, 97, 98, 106, 117, 120, 124, 129, 136, 137, 143, 156, 161, 163, 171, 172, 185, 199, 205, 209, 222, 238, 244, 261 and 262, at least one amino acid substitution selected from the group consisting of S3T, V4I, R45E, R45D, R45Q, P55N, T58W, T58Y, T58L, Q59D, Q59M, Q59N, Q59T, G61D, G61R, S87E, G97S, A98D, A98E, A98R, S106A, S106W, N117E, H120V, H120D, H120K, H120N, S124M, P129D, E136Q, Q137H, S143W, S156D, S161T, S163A, S163G, Y171L, A172S, N185Q, V199M, V205I, Y209W, M222Q, N238H, V244T, N261T and L262N, L262Q, L262D, L262E; wherein the amino acid substitution combination of group (ii) is preferably selected from the group consisting of N238E-L262E, S156D-L262E, S3T-V4I-V205I, S3T-V4I-A228V, G195E-V199M, H120D-S163G-N261D, N76D-A228V-N261D, S3T-N76D-S156D-Y209W, Q137H-S141H-R145H-N238E-L262E, Q137H-S141H-R145H-S156D-L262E, N76D-Q137H-S141H-R145H-A228V-N261D, N76D-Q137H-S141H-R145H-S163G-N238E, H120D-Q137H-S141H-R145H-S163G-N261D, S3T-N76D-Q137H-S141H-R145H-S156D-Y209W;
b) a protease that has proteolytic activity and comprises an amino acid sequence which is at least 70% identical, and with increasing preference at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5% and 98% identical to the amino acid sequence given in SEQ ID NO:2 over its entire length and, in each case based on the numbering according to SEQ ID NO:3 has, (i) at the positions corresponding to positions 9, 130, 133, 144, 217, 252 and 271, at least one amino acid substitution selected from the group consisting of P9T, N130D, N130V, T133A, N144K, Y217M, N252T and Q271E, and (ii) at at least one of the positions corresponding to positions 6, 61, 62, 63, 89, 99, 101, 131, 156, 166, 170, 187, 188, 189, 211 and 224, at least one amino acid substitution selected from the group consisting of Y6F, Y6W, F61G, Q62N, S63Q, S89A, S89G, N99H, D101S, D101E, D101A, G131H, G131Y, G131F, S156R, G166A, G166M, G166L, G166I, K170R, K170G, N187D, N188G, S189T, S189L, S189I, S189R, S211N, S211Q, S224A, S224G, wherein the protease preferably has an amino acid substitution combination selected from the group consisting of P9T-N130D-T133A-N144K-G166M-S189T-Y217M-N252T-Q271E, P9T-N130D-T133A-N144K-G166M-S189T-Y217M-S224A-N252T-Q271E, P9T-N130D-T133A-N144K-G166M-S211N-Y217M-N252T-Q271E, P9T-S89A-N130D-G131H-T133A-N144K-S189T-Y217M-S224A-N252T-Q271E, P9T-S89A-N130D-T133A-N144K-S189T-Y217M-S224A-N252T-Q271E, P9T-S89A-N130D-T133A-N144K-S189T-Y217M-S224A-N252T-Q271E, P9T-S89A-N130D-T133A-N144K-N187D-Y217M-S224A-N252T-Q271E, P9T-S89A-N130D-T133A-N144K-S189R-Y217M-S224A-N252T-Q271E, P9T-S89A-N130D-T133A-N144K-N187D-S189R-Y217M-S224A-N252T-Q271E, P9T-S89A-D101S-N130D-T133A-N144K-S189T-Y217M-S224A-N252T-Q271E, P9T-S89A-D101E-N130D-T133A-N144K-S189T-Y217M-S224A-N252T-Q271E, P9T-S89A-D101A-N130D-T133A-N144K-S189T-Y217M-S224A-N252T-Q271E, P9T-S89A-N99H-N130D-T133A-N144K-K170R-S189T-Y217M-S224A-N252T-Q271E, P9T-S89A-N130D-T133A-N144K-S156R-S189T-Y217M-S224A-N252T-Q271E, P9T-S63Q-S89A-N130D-T133A-N144K-G166A-S189T-Y217M-S224A-N252T-Q271E, P9T-F61 G-Q62-N-S89A-N130D-T133A-N144K-N188G-S189T-Y217M-S224A-N252T-Q271E, Y6W-P9T-N130D-T133A-N144K-S189T-Y217M-S224A-N252T-Q271E, Y6W-P9T-S89A-N130D-T133A-N144K-S189T-Y217M-S224A-N252T-Q271E, Y6W-P9T-F61 G-Q62N-S89A-N130D-T133A-N144K-S189T-Y217M-S224A-N252T-Q271E, Y6W-P9T-S89A-N130D-T133A-N144K-N188G-S189T-Y217M-S224A-N252T-Q271E;
c) a protease that has proteolytic activity and comprises an amino acid sequence which is at least 70% identical, and with increasing preference at least 71%, 72%, 73%, 74%, 75%, 76%, 77%, 78%, 79%, 80%, 81%, 82%, 83%, 84%, 85%, 86%, 87%, 88%, 89%, 90.5%, 91%, 91.5%, 92%, 92.5%, 93%, 93.5%, 94%, 94.5%, 95.5%, 96%, 96.5%, 97%, 97.5%, 98%, 98.5% or 98.8% identical to the amino acid sequence given in SEQ ID NO:3 over its entire length and, in each case based on the numbering according to SEQ ID NO:3 has, (i) at the positions corresponding to positions 3, 4, 101 and 205, the amino acid substitutions S3T, V4I, R101E and V205I, and (ii) at at least one of the positions corresponding to positions 76, 138, 145, 156, 166, 167, 169, 177, 187, 189, 191, 206, 209, 215, 218 or 262, at least one amino acid substitution selected from the group consisting of N76D, N76E, N76Q, A138Q, R145L, R145W, R145Y, S156D, S156Q, S166G, Y167T, A169G, V177L, A187D, F189R, Q191R, Q206A, Q206L, Q206S, Q206T, Y209K, Y209V, Y209W, A215K, A215W, N218S, N218T and L262D, L262E and L262Q, wherein the protease preferably has an amino acid substitution combination selected from the group consisting of S3T-V4I-R101E-V205I-Q206L-Y209W, S3T-V4I-R101E-V205I-N218S, S3T-V4I-R101E-V205I-N76D, S3T-V4I-R101E-V205I-S156D-L262E, S3T-V4I-R101E-V205I-Q206L-Y209W-S156D-L262E, S3T-V4I-R101E-V205I-N76D-Q206L-Y209W, S3T-V4I-R101E-V205I-N76D-S156D-Q206L-Y209W-L262E, S3T-V4I-R101E-V205I-N76D-N218S, S3T-V4I-R101E-V205I-N76D-S156D-Y209W-L262E, S3T-V4I-R101E-V205I-N76D-Y209W, S3T-V4I-R101E-V205I-N76D-S156D-Q206L-L262E, S3T-V4I-R101E-V205I-N76D-Q206L, S3T-V4I-R101E-V205I-S156D-Q206L-Y209W, S3T-V4I-R101E-V205I-Q206L-Y209W-L262E, S3T-V4I-R101E-V205I-A138Q-R145W-Y167T-Q206L, S3T-V4I-R101E-V205I-N76D-R145Y-A215W-N218S-L262E, S3T-V4I-R101E-V205I-N76D-S156D-Y209W-L262E; S3T-V4I-R101E-V205I-Q206L-Y209W-A215K-S156D-L262E, S3T-V4I-R101E-V205I-S156D-S166G-Q191R-Q206L-Y209W-L262E, S3T-V4I-R101E-V205I-S156D-A187D-F189R-Q206L-Y209W-L262E and S3T-V4I-R101E-V205I-A138Q-S156D-V171L-Q206L.

With regard to the cleaning performance achieved, in particular even at high water hardness or high loading, it has proven to be advantageous if the active protease protein has proteolytic activity and comprises an amino acid sequence which has at least 70% sequence identity with the amino acid sequence given in SEQ ID NO:1 over its entire length and, in each case based on the numbering according to SEQ ID NO:3 has, (i) at at least two, preferably three, of the positions corresponding to positions 3, 4, 101 or 205, at least two amino acid substitutions, preferably three amino acid substitutions, selected from the amino acid substitutions S3T, V4I, R101E and V205I, and (ii) at at least one of the positions corresponding to positions 76, 138, 145, 156, 166, 167, 169, 177, 187, 189, 191, 206, 209, 215, 218 or 262, at least one further amino acid substitution, in particular selected from N76D, N76E, N76Q, A138Q, R145L, R145W, R145Y, S156D, S156Q, S166G, Y167T, A169G, V177L, A187D, F189R, Q191R, Q206A, Q206L, Q206S, Q206T, Y209K, Y209V, Y209W, A215K, A215W, N218S, N218T, L262D, L262E and L262Q.

The proteases according to the invention exhibit enzymatic activity, i.e. they are capable of hydrolyzing peptides and proteins, in particular in a washing or cleaning agent. A protease according to the invention is therefore an enzyme that catalyzes the hydrolysis of amide/peptide bonds in protein/peptide substrates and is thereby capable of cleaving proteins or peptides. Furthermore, a protease according to the invention is preferably a mature protease, i.e. the catalytically active molecule without a signal peptide/signal peptides and/or a propeptide/propeptides. Unless otherwise stated, the sequences indicated also refer to mature (processed) enzymes in each case.

Methods for determining protease activity are familiar to and routinely used by a person skilled in the art in the field of enzyme technology. For example, such methods are disclosed in Tenside, volume 7 (1970), pp. 125-132. Alternatively, the protease activity can be determined via the release of the chromophore para-nitroaniline (pNA) from the substrate suc-L-Ala-L-Ala-L-Pro-L-Phe-p-nitroanilide (AAPF). The protease cleaves the substrate and releases pNA. The release of the pNA causes an increase in absorbance at 410 nm, the time profile of which is a measure of the enzymatic activity (cf. Del Mar et al., 1979). The measurement is carried out at a temperature of 25 °C, a pH of 8.6 and a wavelength of 410 nm. The measurement time is 5 min and the measurement interval is 20 s to 60 s. The protease activity is usually expressed in protease units (PU). Suitable protease activities amount to 2.25, 5 or 10 PE per mL of washing liquor, for example. However, protease activity is not equal to zero.

An alternative test for determining the proteolytic activity of the proteases according to the invention is an optical measurement method, preferably a photometric method. The test suitable for this purpose comprises protease-dependent cleavage of the substrate protein casein. The casein is cleaved by the protease into a plurality of smaller partial products. The entirety of these partial products has an increased absorption at 290 nm with respect to non-cleaved casein, wherein this increased absorption can be determined using a photometer, and thus a conclusion can be drawn regarding the enzymatic activity of the protease.

The protein concentration can be determined using known methods, for example the BCA method (bicinchoninic acid; 2,2′-bichinolyl-4,4′-dicarboxylic acid) or the Biuret method (Gornall at al., J. Biol. Chem. 177 (1948): 751-766). In this regard, the active protein concentration can be determined via titration of the active centers using a suitable irreversible inhibitor and determination of the residual activity (cf. Bender et al., J. Am. Chem. Soc. 88, 24(1966): 5890-5913).

In various embodiments of the invention, the protease is a free enzyme. This means that the protease can act directly with all components of an agent and, if the agent is a liquid agent, the protease is directly in contact with the solvent of the agent (e.g., water). In other embodiments, an agent may contain proteases that form an interaction complex with other molecules or that contain a “coating.” In this case, one or more protease molecules can be separated from the other constituents of the agent by a structure surrounding them. Such a separating structure can arise due to, but is not limited to, vesicles, such as a micelle or a liposome. However, the surrounding structure may also be a virus particle, a bacterial cell or a eukaryotic cell. In various embodiments, an agent may contain cells of Bacillus pumilus or Bacillus subtilis which express the proteases according to the invention, or cell culture supernatants of such cells.

In connection with the present invention, the feature whereby a protease has the given substitutions means that it contains one (of the given) substitution(s) at the relevant position, i.e., at least the given positions are not otherwise mutated or deleted—for example, by fragmentation of the protease. In various embodiments, the proteases described herein, with the exception of the explicitly mentioned substitutions, have the sequence of SEQ ID NO:1, i.e., apart from the substituted positions, they are 100% identical to the sequence according to SEQ ID NO:1.

The identity of nucleic acid or amino acid sequences is determined by a sequence comparison. This sequence comparison is based on the BLAST algorithm established and commonly used in the prior art (cf. e.g., Altschul et al. (1990) “Basic local alignment search tool,” J. Mol. Biol., 215:403-410, and Altschul et al. (1997): “Gapped BLAST and PSI-BLAST: a new generation of protein database search programs,” Nucleic Acids Res., 25:3389-3402) and occurs in principle by similar sequences of nucleotides or amino acids in the nucleic acid or amino acid sequences being assigned to one another. A tabular assignment of the relevant positions is referred to as an alignment. A further algorithm available in the prior art is the FASTA algorithm. Sequence comparisons (alignments), in particular multiple sequence comparisons, are created using computer programs. The Clustal series (cf. e.g., Chenna et al. (2003): “Multiple sequence alignment with the Clustal series of programs,” Nucleic Acid Res. 31:3497-3500), T-Coffee (cf. e.g., Notredame et al. (2000): “T-Coffee: A novel method for multiple sequence alignments,” J. Mol. Biol., 302:205-217) or programs based on these programs or algorithms, for example, are frequently used. Also possible are sequence comparisons (alignments) using the computer program Vector NTI® Suite 10.3 (Invitrogen Corporation, 1600 Faraday Avenue, Carlsbad, California, USA) with the specified standard parameters, the AlignX module of which for the sequence comparisons is based on ClustalW, or Clone Manager 10 (use of the scoring matrix BLOSUM 62 for sequence alignment at amino acid level). Unless stated otherwise, the sequence identity indicated herein is determined using the BLAST algorithm.

Such a comparison also allows a conclusion to be drawn about the similarity of the compared sequences to one another. It is usually given in percent identity, i.e., the proportion of identical nucleotides or amino acid radicals at the same positions or positions corresponding to one another in an alignment. In the case of amino acid sequences, the broader concept of homology takes conserved amino acid exchanges into account, i.e., amino acids having similar chemical activity, because these usually perform similar chemical activities within the protein. Therefore, the similarity of the compared sequences can also be indicated as percent homology or percent similarity. Identity and/or homology information can be provided regarding whole polypeptides or genes or only regarding individual regions. Homologous or identical regions of different nucleic acid or amino acid sequences are therefore defined by matches in the sequences. Such regions often have identical functions. They can be small and comprise only a few nucleotides or amino acids. Often, such small regions perform essential functions for the overall activity of the protein. It may therefore be expedient to relate sequence matches only to individual, optionally small, regions. Unless otherwise stated, however, identity or homology information in the present application relates to the entire length of the particular nucleic acid or amino acid sequence indicated.

In the context of the present invention, the indication that an amino acid position corresponds to a numerically designated position in SEQ ID NO:1 therefore means that the corresponding position is associated with the numerically designated position in SEQ ID NO:1 in an alignment as defined above. Furthermore, the assignment of the positions is based on the mature protein. This assignment is also to be used in particular when the amino acid sequence of a protease according to the invention comprises a higher number of amino acid radicals than the protease according to SEQ ID NO:1. Proceeding from the stated positions in the amino acid sequence of the protease according to SEQ ID NO:1, the alteration positions in a protease according to the invention are those which are assigned to said positions in an alignment. In the present application, unless otherwise indicated, the numbering of any sequence changes is based on the numbering according to SEQ ID NO:3.

In addition to the amino acid alterations explained above, proteases according to the invention can have further amino acid alterations, in particular amino acid substitutions, insertions or deletions. Such proteases are developed, for example, by targeted genetic alteration, i.e., by means of mutagenesis methods, and optimized for specific use purposes or with regard to specific properties (for example with regard to their catalytic activity, stability, etc.). Furthermore, nucleic acids according to the invention can be introduced into recombination approaches and thus used to produce completely novel proteases or other polypeptides. The aim is to introduce targeted mutations such as substitutions, insertions or deletions into the known molecules in order, for example, to improve the cleaning performance of enzymes according to the invention. For this purpose, in particular the surface charges and/or the isoelectric point of the molecules and thus their interactions with the substrate can be altered. For example, the net charge of the enzymes can be altered in order to influence the substrate binding in particular for use in washing and cleaning agents. Alternatively or additionally, the stability or catalytic activity of the protease can be increased by one or more corresponding mutations and its cleaning performance can thereby be improved. Advantageous properties of individual mutations, e.g., individual substitutions, can complement one another. A protease already optimized with regard to certain properties, for example with regard to its stability during storage, can therefore additionally be developed in the context of the invention.

For the description of substitutions that relate to exactly one amino acid position (amino acid exchanges), the following convention is applied herein: first, the naturally present amino acid is referred to in the form of the internationally used single-letter code, followed by the associated sequence position and finally the inserted amino acid. Several or alternative exchanges within the same polypeptide chain are separated by slashes. “130D/V” thus means that position 130 has mutated to D or V. In the case of insertions, additional amino acids are named according to the sequence position. In the case of deletions, the missing amino acid is replaced by a symbol, for example a star or a dash, or a Δ is indicated before the corresponding position. For example, P9T describes the substitution of proline at position 9 by threonine, P9TH describes the insertion of histidine following the amino acid threonine at position 9 and P9* or ΔP9 describes the deletion of proline at position 9. This nomenclature is known to a person skilled in the art in the field of enzyme technology.

In addition to the active protease protein, the second detergent composition comprises a liquid carrier as an essential further component. The proportion by weight of the liquid carrier with respect to the total weight of the second detergent composition, based on the total weight thereof, is from 80 to 96 wt. %, preferably from 85 to 95 wt. % and in particular from 85 to 90 wt. %.

The aqueous carrier of the second detergent composition preferably comprises an aqueous-organic solvent mixture. Particular preference is given to the use of organic solvents from the group of ethanol, n-propanol, i-propanol, butanols, glycol, propanediol, butanediol, methylpropanediol, glycerol, diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol monomethyl ether, dipropylene glycol monoethyl ether, methoxytriglycol, ethoxytriglycol, butoxytriglycol, 1-butoxyethoxy-2-propanol, 3-methyl-3-methoxybutanol, propylene-glycol-t-butyl ether, di-n-octyl ether and mixtures thereof, in particular from the group of propanediol, glycerol and ethanol.

The active protease protein in the second detergent composition can be combined with at least one reversible inhibitor compound selected from the group consisting of polyols, in particular glycerol and 1,2-propylene glycol, benzamidine hydrochloride, borax, boric acids, boronic acids or the salts or esters or derivatives thereof, in particular phenylboronic acid derivatives or 4-formylphenylboronic acid (4-FPBA), compounds of formula (I) or (II)

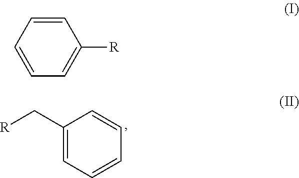

where R is in each case selected from —COOH, C₁₋₆ alkyl-substituted or unsubstituted C₂₋₆ dicarboxylic acids, C₁₋₆ alkyl-substituted or unsubstituted C₂₋₆ carboxylic acids and —OOC—NR²₂, where R² are the same or different and selected from C₁₋₆ alkyl or H, and salts, esters or derivatives thereof, preferably benzoic acid, phenylmalonic acid, benzylmalonic acid, phenylsuccinic acid, benzylsuccinic acid, methyl 3-benzoylpropionate, (S)-3-phenylbutyric acid and benzyl carbamate, and combinations thereof, in order to further increase the stability of the protease in washing and cleaning agents.

The term “phenylboronic acid derivative” is understood to mean a compound of formula (III).

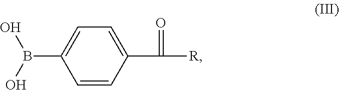

where R is hydrogen, a hydroxyl group, a C₁₋₆ alkyl group, a substituted C₁₋₆ alkyl group, a C₁₋₆ alkenyl, or a substituted C₁₋₆ alkenyl group. Preferably, the functional group R in the phenylboronic acid derivative is a C₁-6 alkyl group and, among these, is more preferably —CH₃, —CH₃CH₂, or —CH₃CH₂CH₂. More preferably, the functional group R in the phenylboronic acid derivative is hydrogen. Particularly preferably, the phenylboronic acid derivative is 4-formylphenylboronic acid (4-FPBA).

The reversible inhibitor compound used can be boric acid.

The reversible inhibitor compound used can be a compound of formula (I) or (II).

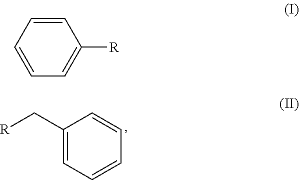

where R is in each case selected from group consisting of —COOH, C₁₋₆ alkyl-substituted or unsubstituted C₂₋₆ dicarboxylic acids, C₁₋₆ alkyl-substituted or unsubstituted C₂₋₆ carboxylic acids and —OOC—NR²₂, where R² are the same or different and selected from C₁₋₆ alkyl or H, and salts, esters or derivatives thereof, and combinations thereof, preferably selected from the group consisting of benzoic acid, phenylmalonic acid, benzylmalonic acid, phenylsuccinic acid, benzylsuccinic acid, methyl 3-benzoylpropionate, (S)-3-phenylbutyric acid and benzyl carbamate.

The second detergent composition is preferably substantially free of boron-containing compounds. “Substantially free of boron-containing compounds” in this context means that the second detergent composition, based on the total weight thereof, contains less than 2 wt. %, preferably less than 1 wt. %, more preferably less than 0.5 wt. %, and particularly preferably less than 0.1 wt. %, of boron-containing compounds. In a very particularly preferred embodiment, the second detergent composition is free of boron-containing compounds, i.e., in particular it does not contain any boron-containing compounds, in particular no boric acid and/or phenylboronic acid derivatives.

In step e) of the method, the second detergent composition is preferably metered from a container integrated into the washing machine, which container is filled with several times the amount of the second detergent composition required to carry out a washing program.

In step e), the second detergent composition is preferably metered such that the second detergent composition is introduced into the aqueous liquor within a period of five minutes, particularly preferably 2 minutes and in particular 1 minute.

The aqueous liquor preferably has a temperature of 26 to 64 °C in step e).

In a preferred embodiment of the method, no bleaching agent and no detergent composition containing bleaching agent is introduced into the laundry treatment chamber of the washing machine before step e). The metering of bleaching agent or detergent compositions containing bleaching agent before step e) has proven to be detrimental to or at least irrelevant for the cleaning effect.

Bleaching agents are in particular understood to mean compounds that supply hydrogen peroxide in an aqueous medium. Among the compounds used as bleaching agents that supply H₂O₂ in water, sodium percarbonate, sodium perborate tetrahydrate, and sodium perborate monohydrate are of particular importance. Further bleaches that can be used are, for example, peroxopyrophosphates, citrate perhydrates, and H₂O₂-supplying peracidic salts or peracids such as persulfates or persulfuric acid. Also useful is urea peroxohydrate percarbamide, which can be described by the formula H₂N—CO—NH₂ H₂O₂. Particularly when the agents are used for cleaning hard surfaces, e.g., for automatic dishwashing, the detergent portion units can, if desired, also contain bleaching agents from the group of organic bleaching agents, although their use is in principle also possible in agents for washing fabrics. Typical organic bleaching agents are diacyl peroxides, such as, for example, dibenzoyl peroxide. Further typical organic bleaches are peroxy acids, wherein alkyl peroxy acids and arylperoxy acids are particularly mentioned as examples. Preferred representatives are (a) peroxybenzoic acid and the ring-substituted derivatives thereof such as alkyl peroxybenzoic acids, but also peroxy-α-naphthoic acid and magnesium monoperphthalate, (b) aliphatic or substituted aliphatic peroxy acids, such as peroxylauric acid, peroxystearic acid, ε-phthalimidoperoxycaproic acid (phthaloiminoperoxyhexanoic acid (PAP)), o-carboxybenzamidoperoxycaproic acid, N-nonenylamidoperadipic acid and N-nonenylamidopersuccinates, and (c) aliphatic and araliphatic peroxydicarboxylic acids, such as 1,12-diperoxycarboxylic acid, 1,9-dipiperoxyazelaic acid, diperoxysebacic acid, diperoxybrassylic acid, diperoxyphthalic acids, 2-decyldiperoxybutane-1,4-diacid, N,N-terephthaloyl-di(6-aminopercaproic acid). Preferred oxygen bleaching agents are formulated in particulate form, wherein the particles have a coating.

After completion of the main wash cycle at the point in time of 100% t_{w}, the aqueous liquor is preferably pumped out of the laundry treatment chamber.

The composition of some preferred first and second detergent compositions for use in the method according to the invention can be found in the following tables (amounts given in wt. % based on the total weight of the solid gel or the casing substance, unless otherwise indicated).

| | | | |
|---|---|---|---|
| Formula 1 | Formula 2 | Formula 3 | Formula 4 |

First detergent composition

| | | | | |
|---|---|---|---|---|
| Surfactant | 50 to 95 | 55 to 92 | 55 to 92 | 60 to 88 |
| Misc. | up to 100 | up to 100 | up to 100 | up to 100 |

Second detergent composition

| | | | | |
|---|---|---|---|---|
| Active protease | 4 to 18 | 4 to 18 | 5 to 15 | 7 to 12 |
| protein | | | | |
| Liquid carrier | 80 to 96 | 80 to 96 | 85 to 95 | 85 to 90 |

| | | | |
|---|---|---|---|
| Formula 5 | Formula 6 | Formula 7 | Formula 8 |

First detergent composition

| | | | | |
|---|---|---|---|---|
| Surfactant | 50 to 95 | 55 to 92 | 55 to 92 | 60 to 88 |
| Misc. | up to 100 | up to 100 | up to 100 | up to 100 |

Second detergent composition

| | | | | |
|---|---|---|---|---|
| Active protease | 4 to 18 | 4 to 18 | 5 to 15 | 7 to 12 |
| protein* | | | | |
| Liquid carrier | 80 to 96 | 80 to 96 | 85 to 95 | 85 to 90 |

| | | | |
|---|---|---|---|
| Formula 11 | Formula 12 | Formula 13 | Formula 14 |

First detergent composition

| | | | | |
|---|---|---|---|---|
| Anionic | 30 to 90 | 30 to 90 | 40 to 80 | 45 to 70 |
| surfactant | | | | |
| Non-ionic | 7 to 50 | 12 to 40 | 12 to 40 | 15 to 30 |
| surfactant | | | | |
| Misc. | up to 100 | up to 100 | up to 100 | up to 100 |

Second detergent composition

| | | | | |
|---|---|---|---|---|
| Active protease | 4 to 18 | 4 to 18 | 5 to 15 | 7 to 12 |
| protein | | | | |
| Liquid carrier | 80 to 96 | 80 to 96 | 85 to 95 | 85 to 90 |

| | | | |
|---|---|---|---|
| Formula 15 | Formula 16 | Formula 17 | Formula 18 |

First detergent composition

| | | | | |
|---|---|---|---|---|
| Anionic | 12 to 40 | 15 to 30 | 15 to 30 | 18 to 25 |
| surfactant | | | | |
| Non-ionic | 12 to 40 | 15 to 30 | 15 to 30 | 18 to 25 |
| surfactant | | | | |
| Misc. | up to 100 | up to 100 | up to 100 | up to 100 |

Second detergent composition

| | | | | |
|---|---|---|---|---|
| Active protease | 4 to 18 | 4 to 18 | 5 to 15 | 7 to 12 |
| protein* | | | | |
| Liquid carrier | 80 to 96 | 80 to 96 | 85 to 95 | 85 to 90 |

* wherein the active protease protein has proteolytic activity and comprises an amino acid sequence which has at least 70% sequence identity with the amino acid sequence given in SEQ ID NO:1 over its entire length and, in each case based on the numbering according to SEQ ID NO:1, has (i) at at least two, preferably three, of the positions that correspond to positions 3, 4, 99 or 199, at least two amino acid substitutions, preferably three amino acid substitutions, selected from the amino acid substitutions 3T, 4I, 99E and 199I, and (ii) at at least one of the positions that correspond to positions 74, 136, 143, 154, 160, 161, 163, 171, 181, 183, 185, 200, 203, 209, 212 or 256, at least one additional amino acid substitution, in particular selected from N74D/E/Q, A136Q, R143L/W/Y, S154D/Q, S160G, Y161T, A163G, V171L, A181D, F183R, Q185R, Q200A/L/S/T, Y203K/V/W, A209K/W, N212S/T and L256D/E/Q.

This application provides, inter alia, the following subjects:
1. A method for washing textiles in a drum washing machine, having the steps of
   a. providing a washing machine with a wash program, including a main wash cycle with the duration t_{w};
   b. introducing textiles into the laundry treatment chamber of the washing machine;
   c. introducing an aqueous liquor into the laundry treatment chamber of the washing machine;
   d. introducing a first detergent composition, comprising at least one surfactant, into the laundry treatment chamber of the washing machine during the main wash cycle at a point in time of 0 to 10% t_{w};
   e. introducing a second detergent composition comprising, based on the total weight thereof,
      i) at least 4 wt. % of active protease protein
      ii) at least 80 wt. % of a liquid carrier
   into the aqueous liquor of the main wash cycle at a point in time of 11 to 99% t_{w}.
2. The washing method according to point 1, wherein the drum washing machine comprises an outer tub, an inner tub attached within the outer tub, as a laundry treatment chamber, and a pumping device that is configured to pump aqueous liquor out of the outer tub.
3. The washing method according to one of the preceding points, wherein the wash program comprises a main wash cycle, at least one rinse cycle, and at least one spin cycle.
4. The washing method according to one of the preceding points, wherein the wash program comprises at least one pre-wash cycle, a main wash cycle, an at least rinse cycle, and an at least spin cycle.
5. The washing method according to one of the preceding points, wherein the duration t_{w} of the main wash cycle is 15 to 400 minutes, preferably 30 to 240 minutes, and in particular 60 to 180 minutes.
6. The washing method according to one of the preceding points, wherein the aqueous liquor in step c) has a volume of 3 to 40 l, preferably of 6 to 30 l, and in particular of 8 to 20 l.
7. The washing method according to one of the preceding points, wherein the aqueous liquor in step c) has a water hardness above 14° dH, preferably above 17°dH and in particular above 21° dH.
8. The washing method according to one of the preceding points, wherein the drum washing machine has a device for detecting the water hardness of the aqueous liquor used in step c), wherein the washing program is preferably configured such that the water hardness detected by means of the device preferably influences the amount of the second detergent composition introduced in step e).
9. The washing method according to one of the preceding points, wherein the water hardness of the aqueous liquor introduced into the laundry treatment chamber of the washing machine in step c) is determined by means of a device for determining the water hardness, and a treatment parameter is determined by comparing the determined water hardness with reference values and treatment parameters assigned to these reference values, which treatment parameter relates to the amount of the first or second detergent composition introduced in step d) and/or step e), and the first detergent composition is introduced in step d), and/or the second detergent composition is introduced in step e), in an amount corresponding to the determined treatment parameter.
10. The washing method according to one of the preceding points, wherein the water hardness of the aqueous liquor introduced into the laundry treatment chamber of the washing machine in step c) is determined by means of a device for determining the water hardness, and a treatment parameter is determined by comparing the determined water hardness with reference values and treatment parameters assigned to these reference values, which treatment parameter relates to the amount of the second detergent composition introduced in step e), and the second detergent composition is introduced in step e) in an amount corresponding to the determined treatment parameter.
11. The washing method according to one of the preceding points, wherein the weight ratio of aqueous liquor to textiles in step c) is above 1:1, preferably above 2:1, and in particular above 5:2.
12. The washing method according to one of the preceding points, wherein the drum washing machine has a device for detecting the weight of the textiles introduced in step b), wherein the washing program is preferably configured such that the textile weight detected by means of the device influences the amount of the second detergent composition introduced in step e).
13. The washing method according to one of the preceding points, wherein the aqueous liquor in step c) has a temperature T₁ of 18 to 25 °C.
14. The washing method according to one of the preceding points, wherein the aqueous liquor is introduced into the laundry treatment chamber of the washing machine before the first detergent composition.
15. The washing method according to one of the preceding points, wherein the aqueous liquor is introduced simultaneously with the first detergent composition into the laundry treatment chamber of the washing machine.
16. The washing method according to one of the preceding points, wherein the aqueous liquor is introduced into the laundry treatment chamber of the washing machine after the first detergent composition.
17. The washing method according to one of the preceding points, wherein the first detergent composition contains, based on the total weight thereof,
   i) 50 to 95 wt. % surfactant
   ii) less than 10 wt. % water.
18. The washing method according to one of the preceding points, wherein the first detergent composition contains, based on the total weight thereof, 55 to 92 wt. %, preferably 60 to 88 wt. %, surfactant.
19. The washing method according to one of the preceding points, wherein the first detergent composition contains anionic surfactant from the group of C₈₋₁₈ alkylbenzenesulfonic acids.
20. The washing method according to one of the preceding points, wherein the first detergent composition contains anionic surfactant from the group of alkyl ether sulfonic acids.
21. The washing method according to one of the preceding points, wherein the first detergent composition contains anionic surfactant from the group of fatty acids.
22. The washing method according to one of the preceding points, wherein the first detergent composition contains, based on the total weight thereof, 30 to 90 wt. %, preferably 40 to 80 wt. % and in particular 45 to 70 wt. %, anionic surfactant.
23. The washing method according to one of the preceding points, wherein the first detergent composition contains nonionic surfactant from the group of the ethoxylated primary C6-18 alcohols, preferably the ethoxylated primary C6-18 alcohols having a degree of alkoxylation≥2, particularly preferably the C12-14 alcohols having 4 EO or 7 EO, the C9-11 alcohols having 7 EO, the C13-15 alcohols having 5 EO, 7 EO or 8 EO, the C13-15 oxo alcohols having 7 EO, the C12-18 alcohols having 5 EO or 7 EO, in particular the C12-18 fatty alcohols having 7 EO or the C13-15 oxo alcohols having 7 EO.
24. The washing method according to one of the preceding points, wherein the first detergent composition contains, based on the total weight thereof, 7 to 50 wt. %, preferably 12 to 40 wt. % and in particular 15 to 30 wt. %, nonionic surfactant.
25. The washing method according to one of the preceding points, wherein the first detergent composition contains polyalkoxylated amine having a weight-average molecular weight Mw in the range from 600 g/mol to 10 000 g/mol, which is obtained by reacting ammonia or primary alkylamines or hydroxyalkylamines having a molecular weight of less than 200 g/mol with alkylene oxides, preferably in proportions by weight of 0.5 to 10 wt. %, preferably 1 to 8 wt. %, and in particular 2 to 6 wt. %.
26. The washing method according to one of the preceding points, wherein the first detergent composition contains polyalkoxylated polyalkylene imine, which is obtained by reacting polyalkylene imines with alkylene oxides, preferably in proportions by weight of 0.5 to 10 wt. %, preferably 1 to 8 wt. %, and in particular 2 to 6 wt. %.
27. The washing method according to one of the preceding points, wherein the first detergent composition contains less than 6 wt. %, and in particular less than 2 wt. %, water.
28. The washing method according to one of the preceding points, wherein the first detergent composition contains, based on the total weight thereof, 0.5 to 29 wt. %, preferably 4.5 to 27 wt. % and in particular 10 to 24 wt. %, organic solvent.
29. The washing method according to one of the preceding points, wherein the first detergent composition contains organic solvent from the group of organic amines, preferably monoethanol amine.
30. The washing method according to one of the preceding points, wherein the first detergent composition contains, based on the total weight thereof,
   i) 30 to 90 wt. %, preferably 40 to 80 wt. %, and in particular 45 to 70 wt. % anionic surfactant from the group of C₈-18 alkylbenzenesulfonic acids, alkyl ether sulfonic acids and fatty acids,
   ii) 7 to 50 wt. %, preferably 12 to 40 wt. %, and in particular 15 to 30 wt. % nonionic surfactant from the group of ethoxylated primary C₆-18 alcohols,
   iii) 0.5 to 29 wt. %, preferably 4.5 to 27 wt. %, and in particular 10 to 24 wt. % organic solvent from the group of organic amines,
   iv) less than 10 wt. %, preferably less than 6 wt. % and in particular less than 2 wt. % water.
31. The washing method according to one of the preceding points, wherein the first detergent composition contains no enzyme.
32. The washing method according to one of the preceding points, wherein, in step d), the first detergent composition is metered from a container integrated into the washing machine, which container is filled with several times the amount of first detergent composition required to run a washing program.
33. The washing method according to one of the preceding points, wherein, in step d), the first detergent composition is introduced into the laundry treatment chamber of the washing machine at a point in time of 0 t_{w}.
34. The washing method according to one of the preceding points, wherein, in step d), the first detergent composition is introduced into the laundry treatment chamber of the washing machine at a point in time of >0 to 10% t_{w}, preferably >0 to 5% t_{w}.
35. The washing method according to one of the preceding points, wherein the second detergent composition comprises, based on the total weight thereof, 4 to 18 wt. %, preferably 5 to 15 wt. % and in particular 7 to 12 wt. % active protease protein.
36. The washing method according to one of the preceding points, wherein the active protease protein has proteolytic activity and comprises an amino acid sequence which has at least 70% sequence identity with the amino acid sequence given in SEQ ID NO:1 over its entire length and, in each case based on the numbering according to SEQ ID NO:1, has (i) at at least two, preferably three, of the positions that correspond to positions 3, 4, 99 or 199, at least two amino acid substitutions, preferably three amino acid substitutions, selected from the amino acid substitutions 3T, 4I, 99E and 199I, and (ii) at at least one of the positions that correspond to positions 74, 136, 143, 154, 160, 161, 163, 171, 181, 183, 185, 200, 203, 209, 212 or 256, at least one additional amino acid substitution, in particular selected from N74D/E/Q, A136Q, R143L/W/Y, S154D/Q, S160G, Y161T, A163G, V171L, A181 D, F183R, Q185R, Q200A/L/S/T, Y203K/V/W, A209K/W, N212S/T and L256D/E/Q.
37. The washing method according to one of the preceding points, wherein the second detergent composition comprises, based on the total weight thereof, 80 to 96 wt. %, preferably 85 to 95 wt. % and in particular 85 to 90 wt. % liquid carrier.
38. The washing method according to one of the preceding points, wherein the second detergent composition comprises an aqueous-organic solvent mixture as liquid carrier.
39. The washing method according to one of the preceding points, wherein the liquid carrier of the second detergent composition comprises organic solvents from the group of ethanol, n-propanol, i-propanol, butanols, glycol, propanediol, butanediol, methylpropanediol, glycerol, diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol mono methyl ether, dipropylene glycol mono ethyl ether, methoxytriglycol, ethoxytriglycol, butoxytriglycol, 1-butoxyethoxy-2-propanol, 3-methyl-3-methoxybutanol, propylene-glycol-t-butylether, di-n-octylether and mixtures thereof, preferably from the group of propanediol, glycerol and ethanol.
40. The washing method according to one of the preceding points, wherein the second detergent composition is substantially free of boron-containing compounds, and is preferably free of boron-containing compounds
41. The washing method according to one of the preceding points, wherein, in step e), the second detergent composition is metered from a container integrated into the washing machine, which container is filled with several times the amount of second detergent composition required to run a washing program.
42. The washing method according to one of the preceding points, wherein, in step e), the second detergent composition is introduced into the aqueous liquor at a point in time of 20 to 70% t_{w}, preferably at a point in time of 25 to 65% t_{w}, particularly preferably at a point in time of 30 to 60% t_{w}.
43. The washing method according to one of the preceding points, wherein, in step e), the second detergent composition is introduced into the aqueous liquor within a period of five minutes, particularly preferably of 2 minutes and in particular of 1 minute.
44. The washing method according to one of the preceding points, wherein the aqueous liquor in step e) has a temperature of 26 to 64 °C.
45. The washing method according to one of the preceding points, wherein no bleaching agent and no detergent composition containing bleaching agent is introduced into the laundry treatment chamber of the washing machine before step e).

1. A method for washing textiles in a washing machine, the method comprising:
providing a wash program to the washing machine, the wash program including a main wash cycle having a duration t_{w};
introducing textiles into a laundry treatment chamber of the washing machine;
introducing an aqueous liquor into the laundry treatment chamber of the washing machine;
introducing a first detergent composition into the laundry treatment chamber of the washing machine during the main wash cycle at a point in time of from 0 to 10% of the duration t_{w} of the main wash cycle, the first detergent composition comprising at least one surfactant; and
introducing a second detergent composition into the laundry treatment chamber of the washing machine during the main wash cycle at a point in time of from 11 to 99% of the duration t_{w} of the main wash cycle, the second detergent composition comprising, based on the total weight of the second detergent composition:
at least 4 wt. % of an active protease protein, and
at least 80 wt. % of a liquid carrier.

2. The method of claim 1, wherein the duration t_{w} of the main wash cycle is from 15 to 400 minutes.

3. The method of claim 1, wherein the aqueous liquor has a water hardness above 14° dH.

4. The method of claim 3, wherein an amount of the second detergent composition introduced into the laundry treatment chamber of the washing machine is determined by comparing the water hardness of the aqueous liquor with reference values having corresponding amounts of the second detergent composition to introduce.

5. The method of claim 1, wherein the first detergent composition comprises, based on the total weight of the first detergent composition:
from 50 to 95 wt. % of a surfactant, and
less than 10 wt. % of water.

6. The method of claim 1, wherein the first detergent composition comprises, based on the total weight of the first detergent composition:
from 30 to 90 wt. % of an anionic surfactant,
from 7 to 50 wt. % of a nonionic surfactant,
from 0.5 to 29 wt. % of an organic solvent, and
less than 10 wt. % of water.

7. The method of claim 1, wherein introducing the first detergent composition is-metered comprises metering the first detergent composition from a container integrated into the washing machine, wherien the container is filled with an amount of the first detergent composition greater than that required by the wash program.

8. The method of claim 1, wherein the second detergent composition comprises an active protease protein in an amount of from 4 to 18 wt. %, based on the total weight of the second detergent composition.

9. The method of claim 8, wherein the active protease protein has proteolytic activity and comprises an amino acid sequence which has at least 70% sequence identity with the amino acid sequence given in SEQ ID NO:1 over its entire length and, in each case based on the numbering according to SEQ ID NO:1, has (i) at at least two of the positions that correspond to positions 3, 4, 99 or 199, at least two amino acid substitutions selected from the amino acid substitutions 3T, 4I, 99E and 199I, and (ii) at least one of the positions that correspond to positions 74, 136, 143, 154, 160, 161, 163, 171, 181, 183, 185, 200, 203, 209, 212 or 256, at least one additional amino acid substitution, selected from N74D/E/Q, A136Q, R143L/W/Y, S154D/Q, S160G, Y161T, A163G, V171L, A181D, F183R, Q185R, Q200A/L/S/T, Y203K/V/W, A209K/W, N212S/T and L256D/E/Q.

10. The method of claim 1, wherein the liquid carrier of the second detergent composition is present in an amount of from 80 to 96 wt. %, based on the total weight of the second detergent composition.

11. The method of claim 1, wherein the liquid carrier of the second detergent composition comprises an aqueous-organic solvent mixture.

12. The method of claim 1, wherein the liquid carrier of the second detergent composition comprises one or more organic solvents selected from the group consisting of ethanol, n-propanol, i-propanol, butanols, glycol, propanediol, butanediol, methylpropanediol, glycerol, diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol mono methyl ether, dipropylene glycol mono ethyl ether, methoxytriglycol, ethoxytriglycol, butoxytriglycol, 1-butoxyethoxy-2-propanol, 3-methyl-3-methoxybutanol, propylene-glycol-t-butylether, di-n-octylether, and mixtures thereof.

13. The method of claim 1, wherein the second detergent composition is substantially free of boron-containing compounds.

14. The method of claim 1, wherein introducing the second detergent composition comprises metering the second detergent composition from a container integrated into the washing machine, wherien the container is filled with an amount of the second detergent composition greater than that required by the wash program.

15. The method of claim 1, wherein the second detergent composition is introduced into the laundry treatment chamber of the washing machine at a point in time of from 20 to 70% of the duration t_{w} of the main wash cycle.

16. The method of claim 1, wherein the washing machine is a drum washing machine.

17. The method of claim 6, wherein:
the anionic surfactant is selected from the group consisting of C8-18 alkylbenzenesulfonic acids, alkyl ether sulfonic acids, and fatty acids,
the nonionic surfactant is selected from the group consisting of ethoxylated primary C6-18 alcohols, and
the organic solvent is selected from the group consisting of organic amines.

18. The method of claim 6, wherein the first detergent composition comprises, based on the total weight of the first detergent composition:
from 45 to 70 wt. % of the anionic surfactant,
from 15 to 30 wt. % of the nonionic surfactant,
from 10 to 24 wt. % of the organic solvent, and
less than 2 wt. % of the water.

19. The method of claim 12, wherein the liquid carrier of the second detergent composition comprises one or more organic solvents selected from the group consisting of propanediol, glycerol, and ethanol.

20. The method of claim 15, wherein the second detergent composition is introduced into the laundry treatment chamber of the washing machine at a point in time of from 30 to 60% of the duration t_{w} of the main wash cycle.


SEQUENCE LISTING (3 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRNGDGQYSWIISGIEWAVANNMDVINMSLGGPNGSTALKNAVDTANNRGVVVVAAAGNSGSTGSTSTVGYPAKYDSTIAVANVNSSNVRNSSSSAGPELDVSAPGTSILSTVPSSGYTSYTGTSMASPHVAGAAALILSKNPNLSNSQVRQRLENTATPLGNSFYYGKGLINAQAASN

SEQ ID NO: 3 - protein, 275 aa, Bacillus sp. [sequence omitted]